(19) United States
(12) Patent Application Publication
(10) Pub. No.: US 2019/0216827 A1
(43) Pub. Date: Jul. 18, 2019

(54) Composite Formulation of Dutasteride and Tadalafil Comprising Glycerol Fatty Acid Ester Derivative or Propylene Glycol Fatty Acid Ester Derivative and Oral Capsule Formulation Comprising the Same

(71) Applicant: Yuyu Pharma, Inc., Jecheon-si, Chungcheongbuk-do (KR)

(72) Inventors: Taegon Baik, Seoul (KR); Seyeon Kim, Suwon-si, Gyeonggi-do (KR); Ju-Hee Kim, Suwon-si, Gyeonggi-do (KR); Seung Han Song, Suwon-si, Gyeonggi-do (KR); Young-Joon Park, Gwacheon-si, Gyeonggi-do (KR)

(21) Appl. No.: 16/099,482

(22) PCT Filed: May 12, 2017
(86) PCT No.: PCT/KR2017/004986

§ 371 Date: Nov. 7, 2018

(30) Foreign Application Priority Data
May 12, 2016 (KR) 10-2016-0058152

(51) Int. Cl. A61K 31/58; A61K 47/14; A61K 31/4985; A61K 9/48; A61K 9/107

(52) U.S. Cl. CPC A61K 31/58; A61K 47/14; A61K 9/107; A61K 9/4858; A61K 31/4985

(57) ABSTRACT

The present invention is related to a composite formulation of dutasteride and tadalafil, therapeutic agents for prostatic hyperplasia, and to an oral capsule preparation containing the same. The composite formulation has the total amount of the composition inside a capsule of 400 mg or less so that the size of the capsule is convenient for the patient to take so as to increase patients' convenience, and includes a glycerol fatty acid ester derivative or a propylene glycol fatty acid ester derivative which may increase the solubility of dutasteride, a poorly soluble medicament and improve the stability of tadalafil so as to dissolve dutasteride and disperse tadalafil.

### TECHNICAL FIELD

The present invention relates to a composite formulation including both of dutasteride and tadalafil, which are poorly soluble medications, and an oral capsule formulation including the same. More particularly, the present invention relates to a composite formulation and an oral capsule formulation including the same, the composite formulation including, as a content liquid of a capsule, a glycerol fatty acid ester derivative or a propylene glycol fatty acid ester derivative which is excellent in solubility of dutasteride, a poorly soluble medication, low in solubility of tadalafil which has a large content, and excellent in stability, so as to dissolve dutasteride and disperse tadalafil, thus minimizing the capsule size, improving medication stability, and improving convenience in oral administration.

### BACKGROUND ART

Prostatic hyperplasia is a pathological term, also referred to as ‘benign prostatic hyperplasia’, which generally refers to the symptoms of voiding dysfunction caused by enlargement of the prostate resulting in increased urethral resistance. In recent times, a numerical score of symptoms that the patients feel, a urinary flow rate representing the intensity of a urinary stream, and the size of the prostate are combined to diagnose patients having results higher than a certain level with prostatic hyperplasia. Prostatic hyperplasia is closely related to aging and male hormones, and 40% to 70% of the age of 60 or older have lower urinary tract symptoms (LUTS) due to enlarged prostate. As such, prostatic hyperplasia is an important problem in an aging society that affects the quality of life. The main causes of prostatic hyperplasia are known as testosterone and aging. In the old age, testosterone levels are lowered, but dihydrotestosterone (DHT), another form of testosterone, is known to cause enlargement of the prostate.

Representative medications used in the treatment of prostatic hyperplasia may include a 5-alpha reductase inhibitor and a phosphodiesterase (PDE) 5 inhibitor. U.S. Pat. No. 5,565,467 discloses a use of dutasteride (chemical name: 17β-N-(2,5-bis (trifluoromethyl)) phenylcarbamoyl-4-aza-5α-androst-1-en-3-one), the 5-alpha reductase inhibitor, represented by formula 1 for the treatment of benign prostatic hyperplasia, prostate cancer and male alopecia. The 5-alpha reductase inhibitor inhibits the conversion of testosterone to DHT, thereby reducing DHT and inhibiting prostate growth.

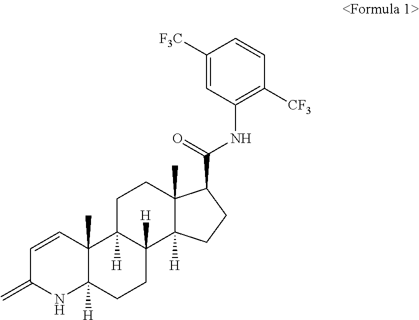

Dutasteride is commercially available under the trade name AVODART®, which is a soft capsule formulation including 0.5 mg of dutasteride. Dutasteride is a medication that is significantly poorly soluble in water, and when dutasteride is administered into the body, the dissolution is low and it is liable to cause absorption problems. Thus, AVODART® has a formulation in which a soft capsule is filled with an oil-based content liquid.

Tadalafil (chemical name: 6R-trans-6-(1,3-benzodioxol-5-yl)-2,3,6,7,12,12a-hexahydro-2-methyl-pyrazino [1′,2′:1,6] pyrido[3,4-b]indole-1,4-dione), the PDE 5 inhibitor, represented by formula 2, was developed as a medication for the treatment of sexual dysfunction, but is used for the treatment of prostatic hyperplasia in the case of the dose of 5 mg only for daily administration.

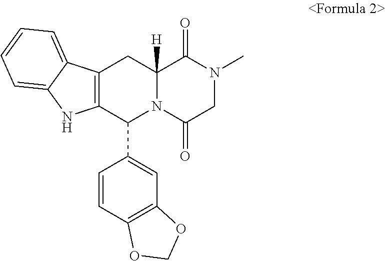 [SMILES: CN1CC(=O)N2[C@H](c3ccc4c(c3)OCO4)c3[nH]c4ccccc4c3C[C@@H]2C1=O]

A combination therapy of the two groups of medications is recommended as an effective treatment modality for moderate to severe lower urinary tract symptoms than monotherapy. The combination therapy is also therapeutically beneficial because the use of PDE 5 inhibitors provides additional benefits to erectile effects without side effects related to sexual function (Therapeutics and Clinical Risk Management, Volume 2015: 11 Pages 507-513).

In order to maximize the treatment of prostatic hyperplasia, a combination of dutasteride and tadalafil is often administered. However, the patients with prostatic hyperplasia are mostly advanced in age, and the number of medicines they take is also large. Considering these points, for convenience of administration, it is desirable to develop a composite medication to reduce the number of medication tablets and reduce the size of the formulation of the composite medication, so as to improve patients' compliance with medication.

To treat benign prostatic hyperplasia, medications should be taken for a long time. In the case of AVODART® which is a soft capsule formulation comprising as a main component, dutasteride, a large amount of oil is used for filling the dutasteride into the soft capsules, thus increasing the size of the soft capsules, and the patients' compliance with medication is low because of the inconvenience of oral administration. When tadalafil is combined to this, the size of the formulation becomes larger, and the patients' compliance may become more problematic. Given that most of the patients with benign prostatic hyperplasia are advanced in age, these problems should not be overlooked.

Since both dutasteride and tadalafil are poorly soluble medications, a large amount of solubilizing agents are needed when the composite formulation of those two are developed as tablets, and the tablet size increases to such an extent that the tablets are too large to be taken. Accordingly, the composite formulation is preferable to be developed into a capsule formulation. In the case of soft capsules, since an amount of tadalafil is more than about 10 times larger than that of dutasteride on average and a large amount of oil is required to prepare capsules containing both medications dissolved therein, the size of soft capsules inevitably increases, causing problems in oral administration. In addition, when tadalafil is dissolved, it may cause stability problems. AVODART® soft capsule, containing a single formulation of dutasteride, has the size of oblong-type No. 6. Accordingly, considering the patients' compliance with the medication, it is necessary to develop a composite formulation with the size of oblong-type No. 6 or less, which is the size of the single formulation.

Accordingly, the present inventors have conducted extensive research to develop a composite formulation of dutasteride and tadalafil minimized in size. As a result, the present inventors have found that when a glycerol fatty acid ester derivative or a propylene glycol fatty acid ester derivative is used as an oil component, the solubility of dutasteride is about 1 mg/ml or higher, and the solubility of tadalafil is about ½ times or less of the solubility of dutasteride. Accordingly, it is possible to develop a composite capsule formulation capable of effectively dissolving dutasteride and dispersing most of tadalafil, and an amount of the content liquid in the capsule may be reduced to about 400 mg or less such that the capsule size may be reduced to oblong-type No. 6 or less. Thus, it is possible to produce a formulation having high patients' compliance with oral administration and excellent in dissolution and bioavailability even in the case of a composite formulation.

Meanwhile, Korean Patent Laid-Open Publication No.10-2014-0108893 also discloses a composite formulation composition containing tadalafil and dutasteride and a method of fabricating the same, which was prepared by mixing a dutasteride preparation with a tadalafil preparation wherein the dutasteride preparation is characterized by comprising a mixed solution, adsorbed on the adsorbent, containing dutasteride, diethylene glycol monoethyl ether, mono/di-glyceride and polyoxyl castor oil; and an adsorbent, and wherein the tadalafil preparation characterized in that a suspension containing tadalafil, a surfactant, a water-soluble polymer and a solvent is made into granules. In the literature, diethylene glycol monoethyl ether, mono/di-glyceride and polyoxyl castor oil are used as an oil component and an adsorbent is coated to fabricate a formulation. However, in the case where a large amount of oil is contained, there is a difficulty in industrialization due to tableting disorder, and because these oils have a low solubility of dutasteride and should be used in a large amount, the size of the composite formulation becomes large and it is disadvantageous for the patients' compliance with oral administration.

On the other hand, when a glycerol fatty acid ester derivative or a propylene glycol fatty acid ester derivative is used as an oil component according to the present invention, a composite formulation having a small size and excellent solubility and enabling easy industrialization may be obtained to overcome such issues described above.

### DISCLOSURE

### Technical Problem

Embodiments of the present invention may be directed to a composite formulation including dutasteride and tadalafil, poorly soluble medications, which is minimized in size and improved in the stability of a medication to enhance patents' compliance, by using a glycerol fatty acid ester derivative or a propylene glycol fatty acid ester derivative as an oil component for a capsular content, and to an oral capsule formation including the composite formulation.

### Technical Solution

According to one embodiment of the present invention, a composite formulation includes: dutasteride represented by formula I; tadalafil represented by formula II; and a fatty acid ester derivative of glycerol or a fatty acid ester derivative of propylene glycol. The fatty acid ester derivative of glycerol or the fatty acid ester derivative of propylene glycol dissolves the dutasteride and disperses the tadalafil.

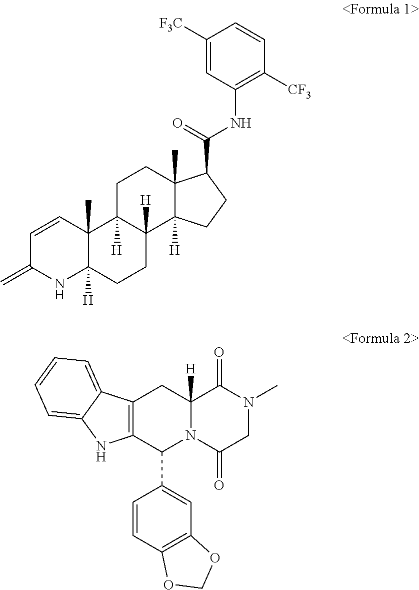

In the fatty acid ester derivative of glycerol or propylene glycol, a fatty acid bonded to glycerol or propylene glycol may have 8 to 18 carbon atoms.

The fatty acid ester derivative of glycerol or propylene glycol may have a solubility of dutasteride of 1.0 mg/ml or higher, and a solubility of tadalafil may be ½ times or less of the solubility of dutasteride.

The fatty acid ester derivative of glycerol or propylene glycol may be at least one selected from the group consisting of glycerol caprylate/caprate, glycerol monooleate, propylene glycol monocaprylate or propylene glycol monolaurate.

A content of the fatty acid ester derivative of glycerol or the fatty acid ester derivative of propylene glycol may be in a range from 79.0 percent by weight (wt %) to 98.95 wt %.

A content of the dutasteride may be in a range from 0.05 wt % to 1.5 wt %, a content of the tadalafil may be in a range from 1 wt % to 20 wt %, and a content of the fatty acid ester derivative of glycerol or propylene glycol may be in a range from 79.0 wt % to 98.95 wt %.

According to another embodiment, an oral capsule formulation includes a composite formulation, the composite formulation including: dutasteride represented by formula I as above; tadalafil represented by formula II as above; and a fatty acid ester derivative of glycerol or a fatty acid ester derivative of propylene glycol. The fatty acid ester derivative of glycerol or propylene glycol dissolves the dutasteride and disperses the tadalafil.

According to still another embodiment, a composite formulation includes: dutasteride represented by Formula I; tadalafil represented by Formula II; a fatty acid ester derivative of glycerol or a fatty acid ester derivative of propylene glycol; and a surfactant. The fatty acid ester derivative of glycerol or propylene glycol dissolves the dutasteride and disperses the tadalafil.

In the fatty acid ester derivative of glycerol or propylene glycol, a fatty acid bonded to glycerol or propylene glycol may have 8 to 18 carbon atoms.

The fatty acid ester derivative may have a solubility of dutasteride of 1.0 mg/ml or higher, and a solubility of tadalafil may be ½ times or less of the solubility of dutasteride.

The fatty acid ester derivative may be at least one selected from the group consisting of glycerol caprylate/caprate, glycerol monooleate, propylene glycol monocaprylate, and propylene glycol monolaurate.

The surfactant may include at least one selected from the group consisting of polyoxyl castor oil, polyoxyl stearic acid, polyoxyl sorbitan fatty acid ester, polyoxyl glyceride, tocopherol polyethylene glycol succinate, and polyoxyethylene-polyoxypropylene copolymers.

A content of the fatty acid ester derivative of glycerol or propylene glycol may be in a range from 49.0 wt % to 97.95 wt %.

A content of the surfactant may be in a range from 1 wt % to 30 wt %.

A content of the dutasteride may be in a range from 0.05 wt % to 1.5 wt %, a content of the tadalafil may be in a range from 1 wt % to 20 wt %, a content of the fatty acid ester derivative of glycerol or the fatty acid ester derivative of propylene glycol may be in a range from 49.0 wt % to 97.95 wt %, and a content of the surfactant may be in a range from 1 wt % to 30 wt %.

According to still yet another embodiment, an oral capsule formulation includes a composite formulation, the composite formulation including: dutasteride represented by formula I as above; tadalafil represented by the above formula II as above; a fatty acid ester derivative of glycerol or a fatty acid ester derivative of propylene glycol; and a surfactant. The fatty acid ester derivative of glycerol or propylene glycol dissolves the dutasteride and disperses the tadalafil.

The composite formulation spontaneously forms an emulsion in vivo after administration.

In the oral capsule formulation, a capsule filling amount of the composite formulation may be in a range from 100 mg to 400 mg.

The foregoing is illustrative only and is not intended to be in any way limiting. In addition to the illustrative aspects, embodiments, and features described above, further aspects, embodiments, and features will become apparent by reference to the drawings and the following detailed description.

### Advantageous Effects

A composite formulation and an oral capsule formulation including the composite formulation according to the present invention may reduce an amount of content liquid to be filled in a capsule, thus minimizing the size of the formulation and accordingly enhancing patients' compliance with oral administration.

### DESCRIPTION OF DRAWINGS

FIG. 1 is a photograph comparing a capsule prepared according to Example 15 with an AVODART® soft capsule of Comparative Example 1;

FIG. 2 is a graph of dissolution tests of Examples 5, 10 and 34, and FIG. 3 is a graph of dissolution tests of Examples 14, 15 and 16; and

FIG. 4 is a pharmacokinetic experiment graph of Example 16.

### MODE FOR INVENTION

Dutasteride and tadalafil are pharmacologically active components and are poorly soluble medications.

A glycerol fatty acid ester derivative and a propylene glycol fatty acid ester derivative solubilize dutasteride to increase the solubility of the medication. In addition, a glycerol fatty acid ester derivative and a propylene glycol fatty acid ester derivative lower the electrostatic force of a surface of tadalafil, another poorly soluble medication having a larger content than that of dutasteride, and enhance dispersion force of tadalafil, rather than dissolving tadalafil, so that tadalafil may be well dispersed in a capsular content liquid while well dissolved in an aqueous solution.

Accordingly, when the composite capsule formulation is prepared using the aforementioned derivatives, the size of a final capsule formulation may be minimized, the patients' compliance with the medication may be enhanced, and the bioavailability may be excellent in that dutasteride and tadalafil, poorly soluble medications, show a dissolution rate of about 85% or more within 60 minutes. Accordingly, it is appreciated that the composite capsule formulation of dutasteride and tadalafil according to the present invention is superior to any conventional formulation in terms of patients' compliance and absorption in vivo.

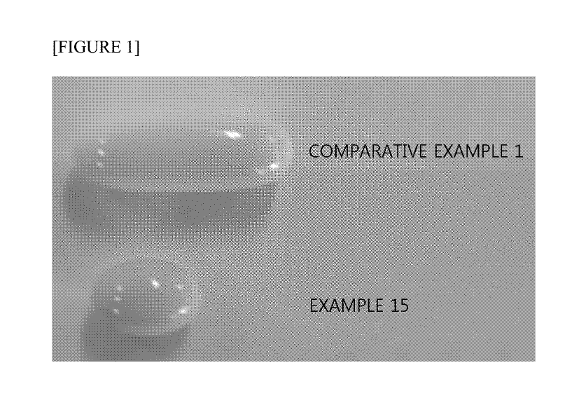

FIG. 1 is a photograph illustrating the characteristics of a capsule prepared according to Example 15. Referring to FIG. 1, it is appreciated that the capsule according to Example 15 is an oval-type No. 2 soft capsule which is minimized in size to half the size of an AVODART® soft capsule which is a single formulation of dutasteride.

In the composite capsule formulation composition according to one embodiment of the present invention, a content liquid may have a phase of a milky white solution and form an emulsion spontaneously in the body by contacting water after oral administration.

The main point in the composite capsule formulation composition of the present invention is contents contained in a hard or soft capsule, wherein dutasteride is dissolved and tadalafil in the form of a liquid suspension evenly dispersed. In order to exhibit such a property, an oil component of the capsular content selects an oil material having a solubility of dutasteride of 1 mg/ml or higher and a solubility of tadalafil of ½ times or less of the solubility of dutasteride to dissolve dutasteride and disperse, rather than dissolving, tadalafil. Accordingly, a composite capsule formulation including both of dutasteride and tadalafil in a desired amount in the capsule with a small amount of a content solution may be provided, and the size of the composite formulation may be minized, thereby providing convenience for patients. In general, the size of capsules, especially soft capsules, suitable for being taken by elderly patients is oblong-type No. 6 or less. If the capsule has the size larger than the above, it is likely to be difficult to be taken, and in the case of a patient suffering from dysphagia, the capsule may be caught within the esophagus when taken. Considering that the majority of patients with prostate problems, which is the treatment area of the present invention, are elderly, that the number of medicines they take is large, and that the large size of the formulation is not easy to swallow, the capsule size of the composite formulation of dutasteride and tadalafil should be designed to be oblong-type No. 6 or less so that the compliance with medication may be enhanced. In order to design the capsule to have the size of oblong-type No. 6 or less, the maximum amount of the capsular content liquid should be 400 mg. In the case of dutasteride which is a main component and has an effective dose ranging from 0.5 mg to 1 mg in the content liquid of 400 mg or less, it is necessary to use an oil component with a solubility of about 1 mg/ml or higher to completely dissolve dutasteride which is poorly soluble. In addition, the oil should well disperse tadalafil which has an effective dose ranging from 5 mg to 20 mg, and allow the two medications to be well dissolved. In respect of dissolution characteristics of the medications, dutasteride may have a high dissolution rate and high bioavailability when dissolved in the content liquid, and tadalafil may be well dissolved in a solution when dissolved in the content liquid or evenly dispersed in the content liquid without electrostatic force. Therefore, it is most important to select such an oil component.

That is, it is critical to select an oil component for the capsular content liquid of the composite formulation, which is capable of dissolving dutasteride, a poorly soluble medication, to improve dissolution properties of dutasteride and increase bioavailability thereof, while eliminating the electrostatic force of tadalafil and enhancing the dispersion property thereof to water to improve dissolution properties of tadalafil.

In order to obtain such properties, it is necessary to use, as a capsule filler, oil having an excellent solubility of dutasteride, e.g., 1 mg/ml, or higher. When the solubility is not sufficient, the size of the formulation becomes large and the patients' compliance with the medication is inevitably degraded. On the other hand, in regard to tadalafil, it has been found that when an oil having a low solubility and good dispersibility is selected, a composite formulation excellent in dissolution and minimized in size may be provided, thus increasing the patients' compliance with medication. The solubility of tadalafil was found to be most suitable when it is ½ times or less of the solubility of dutasteride. Considering that the therapeutic dose of dutasteride is 0.5 mg and that the minimum dose of tadalafil is 5 mg, in order for the solubility of dutasteride to be 1 mg/ml, or higher, and in order for the solubility of tadalafil to be ½ times or less of the solubility of dutasteride, the solubility of dutasteride has to be 20 times or more of the solubility of tadalafil based on the therapeutic dose. From the above, it could be known that the oil, which provides tadalafil and dutastride with such solubility and which gives superiority in dissolution of the two medications, is the most eligible for the capsular content. Accordingly, by selecting oil having the solubility of tadalafil which is ½ times or less of the solubility of dutasteride among oils having the solubility of dutasteride of 1 mg/ml, or higher, a composite formulation that may dissolve dutasteride and disperse tadalafil, rather than dissolving tadalafil, may be provided and the size of the composite formulation may be minimized.

As an example of such components, a glycerol fatty acid ester derivative or a propylene glycol fatty acid ester derivative is preferable. A glycerol fatty acid ester derivative or a propylene glycol fatty acid ester derivative in which a fatty acid used for the ester derivatives has 8 to 18 carbon atoms is more preferable. In particular, glycerol caprylate/caprate, glycerol monooleate, propylene glycol monocaprylate, propylene glycol caprylate/caprate, propylene glycol monolaurate, and a combination thereof are most preferable, but the present invention is not limited thereto. The content of the glycerol fatty acid ester derivative or the propylene glycol fatty acid ester derivative is preferably in a range of 79.0 percent by wt % to 98.95 wt % with respect to the total weight of the content liquid filled in the capsule. When the content is less than 79 wt %, the size of the capsule increases and the capsule is difficult for the patient to take. When the content is more than 98.95 wt %, the content of dutasteride and tadalafil, the medicinal components, becomes less and it may be difficult for the medicament to exhibit its medicinal effects.

Experimental example 1 is a result of comparing solubilities of dutasteride and tadalafil according to the type of oils. As shown in Table 13 of Experimental example 1, in the case of the glycerol fatty acid ester derivative and the propylene glycol fatty acid ester derivative having 8 to 18 carbon atoms which are used as a capsule filling agent of the composite formulation according to the present invention, the solubility of dutasteride is 1 mg/ml, or higher such that dutasteride may be completely dissolved in a soft capsule of the size of oblong-type No. 6 or less, while the solubility of tadalafil is ½ times or less of the solubility of dutasteride such that tadalafil may be dispersed therein, rather than being dissolved, and accordingly, the size of the formulation may be substantially minimized. When an oil component having the solubility of tadalafil that exceeds ½ of the solubility of dutasteride is used, a part of tadalafil is dispersed in the content liquid in the capsule while another part thereof is present in a dissolved form, and the tadalafil partly dissolved in the oil is reprecipitated by the temperature drop in winter and the particle size and electrostatic force increase during precipitation and the dissolution rate may decrease. According to the results of Experimental example 1, when a glycerol fatty acid ester derivative or a propylene glycol fatty acid ester derivative is used as the oil component of the composite formulation, although a small amount of oil is used, a reduced-sized formulation, convenient for oral administration, in which dutasteride is dissolved and tadalafil is dispersed, rather than being dissolved, may be provided, and in terms of dissolution, a dissolution rate is close to 100%.

According to the present invention, an antioxidant such as ascorbic acid, butylhydroxyanisol, butylhydroxytoluene and sulfuric acid sulfate may be used to enhance the oxidative stability of the glycerol fatty acid ester derivative or the propylene glycol fatty acid ester derivative, and butylhydroxytoluene may be preferably used. It is preferable that the content of such antioxidants be 1% or less with respect to the total amount of the content liquid of the capsule formulation.

The composition of the composite formulation according to the present invention may include a surfactant in the content liquid. When the capsule is disintegrated and the surfactant contacts the gastrointestinal fluid in vivo, the surfactant serves to stably emulsify the glycerol fatty acid ester derivative or the propylene glycol fatty acid ester derivative to form a transparent emulsion, thereby accelerating the dissolution rate of dutasteride and tadalafil. Examples of the surfactant capable of stably emulsifying the glycerol fatty acid ester derivative or the propylene glycol fatty acid ester derivative may include polyoxyethylene-polyoxypropylene copolymers (e.g., poloxamer 407 and poloxamer 124), polyethylene glycol-15-hydroxystearate (e.g., solutol HS 15), sucrose fatty acid ester, synthetic vitamin E derivatives (e.g., vitamin E TPG), polyoxyethyleneglycolated natural or hydrogenated castor oil (e.g., cremophor RH 40 and cremophor RH 60), sorbitan fatty acid ester (e.g., span 80), polyoxyethylene sorbitan fatty acid ester (e.g., polysorbate 80 and polysorbate 20), polyoxyethylene alkyl ester (e.g., brij 52), polyoxyethylene stearate (e.g., myrj 52), fatty acid macrogol glycerides (e.g., gelucire 44/14), polyglyceryl fatty acid ester (e.g., plurol oleique), lecithin and glyceryl fatty acid ester (e.g., glyceryl monostearate). Among them, polyethylene glycol-15-hydroxystearate, polyoxyethyleneglycolated natural or hydrogenated castor oil, polyoxyethylene-polyoxypropylene copolymers, synthetic vitamin E derivatives, sorbitan ester, polyoxyethylene sorbitan fatty acid ester, and polyoxyethylene alkyl ester may be preferably used.

The surfactant may be one or more selected from the group consisting of, for example, polyoxylated castor oil (e.g., polyoxyl 35 cured castor oil, polyoxyl 35 castor oil, polyoxyl 40 cured castor oil, polyoxyl 50 cured castor oil, polyoxyl 60 cured castor oil and polyoxyl cured castor oil), polyoxyl stearic acid, polyoxyl sorbitan fatty acid ester, polyoxyl glyceride, tocopherol polyethylene glycol succinate and polyoxyethylene-polyoxypropylene copolymers, but the present invention is not limited thereto.

The content of the surfactant is preferably in a range such that the glycerol fatty acid ester derivative or the propylene glycol fatty acid ester derivative may be stably emulsified to form the transparent emulsion and such that the properties of a capsule shell may not be changed. The content is preferably 1 wt % to 30 wt %, and more preferably 5 wt % to 20 wt %, with respect to the total weight of the content. When the content thereof is less than 1 wt %, the emulsifying action may be weak, and when the content is more than 30 wt %, the capsule shell may harden and the disintegration of the capsule may be delayed, resulting in poor dissolution.

One embodiment of the present invention provides an oral capsule formulation filled with the composite formulation composition including dutasteride, tadalafil, a glycerol fatty acid ester derivative or a propylene glycol fatty acid ester derivative as a content liquid filler, and a surfactant.

The composition may be prepared into a soft capsule formulation, by means of gelatin or succinic acid gelatin, a plasticizer (glycerin and sorbitol) and a disintegration aid (glycine and citric acid), which are typically used as capsule bases, with the use of a conventional rotary type automatic charger.

In addition, a capsule may be prepared by charging the composite formulation into a hard capsule with a hard capsule filling machine for liquid filling. Examples of the base used in the hard capsule may include gelatin/hydroxypropylmethylcellulose, and a plasticizer (glycerin, citric acid, sorbitol solution and glycine).

The content liquid, the composite formulation composition according to the present invention, to be filled in the capsule, completely dissolves dutasteride and disperses tadalafil with the glycerol fatty acid ester derivative or the propylene glycol fatty acid ester derivative as an oily phase and is filled in a soft capsule or a hard capsule to form a capsule. The preparation of the content liquid may be controlled according to various pharmaceutical manufacturing processes. For example, dutasteride may be dissolved by stirring a glycerol fatty acid ester derivative or a propylene glycol fatty acid ester derivative, tadalafil may be added thereto while vigorously dispersing the solution (e.g., by means of a propeller-containing mixer, a homogenizer, a microfluidizer, a high pressure homogenizer, and an ultrasonic vibrator), thus forming a suspension, and the suspension may be used as the content liquid. The content liquid may be prepared into a soft capsule or a hard capsule by the above-described method.

Hereinafter, the composite formulation according to embodiments of the present invention will be described in detail with reference to examples and comparative examples. However, this does not limit the scope of the present invention.

### EXAMPLES 1 AND 2

To a 2 l preparation container equipped with a stirrer, 1.094.9 g of oil was added as shown in Table 1, and while intensely stirring the mixture, 5 g of dutasteride was slowly added thereto. After the dutasteride was completely dissolved, 0.1 g of butylhydroxytoluene as an antioxidant, was added thereto and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion, such that a composite formulation of dutasteride and tadalafil was prepared.

Separately, a soft capsule shell having the composition indicated in Table 2 was prepared. First, concentrated glycerin and a D-sorbitol solution were put into a gelatin preparation tank. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger, and 115 mg of the prepared composite formulation content was charged into an oval-type No. 2 soft capsule to a total weight of 235 mg by using a rotary automatic charger. Then, drying and screening processes were performed such that an oral soft capsule formulation was prepared.

TABLE 1

| | Component | Example 1 | | Example 2 | |
|---|---|---|---|---|---|
| Main | Dutasteride | 5 | g | 5 | g |
| component | Tadalafil | 50 | g | 50 | g |
| Oil | Glyceryl monocaprate | 1,094.9 | g | — | |
| | Glycerol monooleate | — | | 1,094.9 | g |
| Antioxidant | butylhydroxytoluene | 0.1 | g | 0.1 | g |

| | | |
|---|---|---|
| Contents per capsule (mg) | 115.0 | 115.0 |

TABLE 2

| Component | Prepared amount | |
|---|---|---|
| Gelatin | 3299.2 | g |
| Concentrated glycerin | 1003.2 | g |
| Disorbitol solution | 428.4 | g |
| Titanium oxide | 29.6 | g |
| Ferric oxide | 3.2 | g |
| Purified water | 2978.8 | g |

### EXAMPLES 3 TO 10

To a 2 l preparation container equipped with a stirrer, oils and surfactants were added respectively in the amounts shown in Table 3 as below, and the mixture was intensely stirred to prepare a transparent solution in which the surfactants are dissolved. After 5 g of dutasteride was slowly added thereto and completely dissolved, butylhydroxytoluene as an antioxidant was added thereto. The mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 4 as below was prepared. First, concentrated glycerin and a D-sorbitol solution were put into a gelatin preparation tank in the composition shown in Table 4 below. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger, and 115 mg of the prepared composite formulation content was charged into an oval-type No. 2 soft capsule to a total weight of 235 mg by using a rotary automatic charger. Then, drying and screening processes were performed such that an oral soft capsule formulation was prepared.

TABLE 3

| | | Example | Example | Example | Example | Example | Example | Example | Example |
|---|---|---|---|---|---|---|---|---|---|
| | Component | 3 | 4 | 5 | 6 | 7 | 8 | 9 | 10 |
| Main | Dutasteride | 5 g | 5 g | 5 g | 5 g | 5 g | 5 g | 5 g | 5 g |
| component | Tadalafil | 50 g | 50 g | 50 g | 50 g | 50 g | 50 g | 50 g | 50 g |
| Oil | Glyceryl | 930.7 g | 985.7 g | 876 g | 930.8 g | — | 985.7 g | — | — |
| | caprylate/caprate | | | | | | | | |
| | Glycerol monooleate | — | — | — | — | 1011.9 g | — | 930.7 g | 876.9 g |
| Surfactant | Polyoxyl 40 hardened | 65.7 g | 43.8 g | — | — | — | 43.8 g | 65.7 g | 87.6 g |
| | castor oil | | | | | | | | |
| | Polysorbate 20 | — | — | — | 65.7 g | — | — | — | — |
| | Polysorbate 80 | — | — | 165 g | — | — | — | — | — |
| | Lauroyl Macrogol | 43.8 g | 29.2 g | — | 43.8 g | — | — | — | — |
| | glyceride | | | | | | | | |
| | Tocopherol polyethylene | — | — | — | — | — | 29.2 g | 43.8 g | 58.4 g |
| | glycol succinate | | | | | | | | |
| | Polyoxyl 15 | — | — | — | — | 54.0 g | — | — | — |
| | hydroxystearate | | | | | | | | |
| | Poloxamer 124 | 54.7 g | 36.2 g | 54 g | — | 29.0 g | 36.2 g | 54.7 g | 72 g |
| | Poloxamer 188 | — | — | — | 54.7 g | — | — | — | — |
| Antioxidant | Butylhydroxytoluene | 0.1 g | 0.1 g | — | — | 0.1 g | 0.1 g | 0.1 g | 0.1 g |

| | | | | | | | | |
|---|---|---|---|---|---|---|---|---|
| Contents per capsule (mg) | 115.0 | 115.0 | 115.0 | 115.0 | 115.0 | 115.0 | 115.0 | 115.0 |

TABLE 4

| Component | Amount | |
|---|---|---|
| Succinic acid gelatin | 3383.2 | g |
| Concentrated glycerin | 968 | g |
| Disorbitol solution | 416 | g |
| Titanium oxide | 29.6 | g |
| Ferric oxide | 3.2 | g |
| Purified water | 2978.8 | g |

### EXAMPLES 11 AND 12

To a 2 l preparation container equipped with a stirrer, 1.094.9 g of oil was added in the amount shown in Table 5 as below, and while intensely stirring the mixture, 5 g of dutasteride was slowly added thereto. After the dutasteride was completely dissolved, 0.1 g of butylhydroxytoluene as an antioxidant was added thereto and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion, such that a composite formulation of dutasteride and tadalafil was prepared.

Separately, a soft capsule shell having the composition shown in Table 4 as above was prepared. First, concentrated glycerin and a D-sorbitol solution were put into a gelatin preparation tank in the composition shown in Table 4 below. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger, and 115 mg of the prepared composite formulation content was charged into an oval-type No. 2 soft capsule to a total weight of 235 mg by using a rotary automatic charger. Then, drying and screening processes were performed such that an oral soft capsule formulation was prepared.

TABLE 5

| | Component | Example 11 | | Example 12 | |
|---|---|---|---|---|---|
| Main | Dutasteride | 5 | g | 5 | g |
| component | Tadalafil | 50 | g | 50 | g |
| Oil | Propylene glycol monocaprylate | 1,094.9 | g | — | |
| | Propylene glycol monolaurate | — | | 1,094.9 | g |
| Antioxidant | Butylhydroxytoluene | 0.1 | g | 0.1 | g |

| | | |
|---|---|---|
| Contents per capsule (mg) | 115.0 | 115.0 |

### EXAMPLES 13 to 23

To a 2 l preparation container equipped with a stirrer, oils and surfactants were added respectively in the amounts shown in Tables 6 and 7 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in Tables 6 and 7 as below and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 2 as above was prepared. First, concentrated glycerin and a D-sorbitol solution were put into a gelatin preparation tank in the composition shown in Table 2. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger, and 115 mg of the prepared composite formulation content was charged into an oval-type No. 2 soft capsule to a total weight of 235 mg by using a rotary automatic charger. Then, drying and screening processes were performed such that an oral soft capsule formulation was prepared.

TABLE 6

| | | Example | | Example | Example | Example | Example |
|---|---|---|---|---|---|---|---|
| | Component | 13 | | 14 | 15 | 16 | 17 |
| Main | Dutasteride | 5 | g | 5 g | 5 g | 5 g | 5 g |
| component | Tadalafil | 50 | g | 50 g | 50 g | 50 g | 50 g |
| Oil | Propylene glycol monolaurate | 1,029.9 | g | 930.7 g | 930.7 g | 875.9 g | 995.9 g |
| Surfactant | Polyoxyl 40 hardened castor | — | | 65.7 g | 65.7 g | 87.6 g | — |
| | oil | | | | | | |
| | Polysorbate 80 | 29 | g | — | — | — | — |
| | Lauroyl Macrogol glyceride | — | | — | — | — | 44.3 g |
| | Tocopherol polyethylene | — | | — | 43.8 g | 58.4 g | 54.7 g |
| | glycol succinate | | | | | | |
| | Polyoxyl 8 capaylocaproyl | — | | 43.8 g | — | — | — |
| | glyceride | | | | | | |
| | Poloxamer1 24 | 36 | g | 54.7 g | 54.7 g | 73 g | — |
| Antioxidant | Butylhydroxytoluene | 0.1 | g | 0.1 g | 0.1 g | 0.1 g | 0.1 g |

| | | | | | |
|---|---|---|---|---|---|
| Contents per capsule (mg) | 115.0 | 115.0 | 115.0 | 115.0 | 115.0 |

TABLE 7

| | | Example | Example | Example | Example | Example | Example |
|---|---|---|---|---|---|---|---|
| | Component | 18 | 19 | 20 | 21 | 22 | 23 |
| Main | Dutasteride | 5 g | 5 g | 5 g | 5 g | 5 g | 5 g |
| component | Tadalafil | 50 g | 50 g | 50 g | 50 g | 50 g | 50 g |
| Oil | Glyceryl caprylate/caprate | — | — | — | 65.7 g | 65.7 g | 87.6 g |
| | Propylene glycol monolaumte | 985 g | 935 g | 985.7 g | 930.7 g | 930.7 g | 876 g |
| Surfactant | Polyoxyl 40 hardened castor | — | 80 g | 43.8 g | — | — | 58.4 g |
| | oil | | | | | | |
| | Polysorbate 20 | — | — | 29.2 g | — | — | — |
| | Polysorbate 80 | 55 g | — | — | — | — | — |
| | Lauroyl Macrogol glyceride | — | — | — | 43.8 g | 43.8 g | — |
| | Tocopherol polyethylene | 55 g | 80 g | — | 54.7 g | 54.7 g | 73 g |
| | glycol succinate | | | | | | |
| | Poloxamer 124 | — | — | 36.2 g | — | — | — |
| Antioxidant | Butylhydroxytoluene | — | — | 0.1 g | 0.1 g | 0.1 g | — |

| | | | | | | |
|---|---|---|---|---|---|---|
| Contents per capsule (mg) | 115.0 | 115.0 | 115.0 | 115.0 | 115.0 | 115.0 |

### EXAMPLES 24 TO 27

To a 3 l preparation container equipped with a stirrer, oils and surfactants were added respectively in the amounts shown in Table 8 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in Table 8 and the mixture was stirred to prepare a transparent solution. After 200 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 4 as above was prepared. First, concentrated glycerin and a D-sorbitol solution were put into a gelatin preparation tank in the composition shown in Table 4. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger. After 160 mg of the prepared composite formulation content was charged into an oval-type No. 3 soft capsule to a total weight of 280 mg by using a rotary automatic charger. Then, drying and screening processes were performed such that an oral soft capsule formulation was prepared.

TABLE 8

| | Component | Example 24 | | Example 25 | | Example 26 | | Example 27 | |
|---|---|---|---|---|---|---|---|---|---|
| Main component | Dutasteride | 5 | g | 5 | g | 5 | g | 5 | g |
| | Tadalafil | 200 | g | 200 | g | 200 | g | 200 | g |

| | | | | | | |
|---|---|---|---|---|---|---|
| Oil | Glyceryl caprylate/caprate | 1,394.9 | g | — | — | — |

| | | | | | | | |
|---|---|---|---|---|---|---|---|
| Propylene glycol monolaurate | — | 1,394.9 | g | 1,315 | g | 1,258.9 | g |

| | | | | | | |
|---|---|---|---|---|---|---|
| Surfactant | Polyoxyl 40 hardened castor oil | — | — | — | 68 | g |
| | Tocopherol polyethylene | — | — | — | 34 | g |

| glycol succinate | | | | | |
|---|---|---|---|---|---|
| Polyoxyl 8 caprylocaproyl | — | — | — | | — |
| glyceride | | | | | |
| Polyoxyl 15 hydroxystearate | — | — | 80.0 | g | — |

| | Poloxamer 124 | — | | — | | — | 34 | g |
|---|---|---|---|---|---|---|---|---|
| Antioxidant | Butylhydroxytoluene | 0.1 | g | 0.1 | g | — | 0.1 | g |

| | | | | |
|---|---|---|---|---|
| Contents per capsule (mg) | 160.0 | 160.0 | 160.0 | 160.0 |

### EXAMPLES 28 TO 31

To a 3 l preparation container equipped with a stirrer, oils and surfactants were added respectively in the amounts shown in Table 9 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in Table 9 and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 4 was prepared. First, concentrated glycerin and a D-sorbitol solution were put into a gelatin preparation tank in the composition shown in Table 4. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger. After 220 mg of the prepared composite formulation content was charged into an oblong-type No. 4 soft capsule to a total weight of 370 mg by using a rotary automatic charger. Then, drying and screening processes were performed such that an oral soft capsule formulation was prepared.

TABLE 9

| | | Example | | Example | | Example | | Example | |
|---|---|---|---|---|---|---|---|---|---|
| | Component | 28 | | 29 | | 30 | | 31 | |
| Main | Dutasteride | 5 | g | 5 | g | 5 | g | 5 | g |
| component | Tadalafil | 50 | g | 50 | g | 50 | g | 50 | g |
| Oil | Glyceryl caprylate/caprate | — | | — | | 129 | g | 129 | g |
| | Glycerol monooleate | 1,822.9 | g | — | | — | | — | |
| | Propylene glycol monolaurate | — | | 1,823 | g | 1,822.9 | g | 1,822.9 | g |
| Surfactant | Polyoxyl 40 hardened castor | 129 | g | 129 | g | 86 | g | 107 | g |
| | oil | | | | | | | | |
| | Polysorbate 80 | — | | — | | — | | 86 | g |
| | Tocopherol polyethylene | — | | 86 | g | 107 | g | — | |
| | glycol succinate | | | | | | | | |
| | Polyoxyl 8 caprylocaproyl | 86 | g | — | | — | | — | |
| | glyceride | | | | | | | | |

| | | | | | | |
|---|---|---|---|---|---|---|
| Poloxamer 124 | 107 | g | 107 | g | — | — |

| | | | | | | | | |
|---|---|---|---|---|---|---|---|---|
| Antioxidant | Butylhydroxytoluene | 0.1 | g | — | 0.1 | g | 0.1 | g |

| | | | | |
|---|---|---|---|---|
| Contents per capsule (mg) | 220.0 | 220.0 | 220.0 | 220.0 |

### EXAMPLES 32 TO 35

To a 5 l preparation container equipped with a stirrer, oils and surfactants were added respectively in the amounts shown in Table 10 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in the Table 10 and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 4 was prepared. First, concentrated glycerin and a D-sorbitol solution were put into a gelatin preparation tank in the composition shown in Table 4. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger. After 350 mg of the prepared composite formulation content was charged into an oblong-type No. 6 soft capsule to a total weight of 600 mg by using a rotary automatic charger. Then, drying and screening processes were performed such that an oral soft capsule formulation was prepared.

TABLE 10

| | Component | Example 32 | | Example 33 | | Example 34 | | Example 35 | |
|---|---|---|---|---|---|---|---|---|---|
| Main component | Dutasteride | 5 | g | 5 | g | 5 | g | 5 | g |
| | Tadalafil | 50 | g | 50 | g | 50 | | 50 | g |

| Oil | Glyceryl caprylate/caprate | — | | — | — | | 198.8 | g |
|---|---|---|---|---|---|---|---|---|
| | Glycerol monooleate | 2,929.3 | g | — | 184 | g | 133.2 | g |

| | | | | | | |
|---|---|---|---|---|---|---|
| Propylene glycol monocaprylate | — | — | 3,020.9 | g | 2,946.4 | g |

| | Propylene glycol monolaurate | — | | 2,929.4 | g | — | | — |
|---|---|---|---|---|---|---|---|---|
| Surfactant | Polyoxyl 40 hardened castor oil | 206.7 | g | 206.7 | g | 240.0 | g | — |

| | | | | | | |
|---|---|---|---|---|---|---|
| Tocopherol polyethylene glycol succinate | — | 136.8 | g | — | 166.5 | g |

| Polyoxyl 8 caprylocaproyl glyceride | 136.8 | g | — | | — | — |
|---|---|---|---|---|---|---|
| Poloxamer 124 | 172.1 | g | 172.1 | g | — | — |

| | | | | | | | | |
|---|---|---|---|---|---|---|---|---|
| Antioxidant | Butylhydroxytoluene | 0.1 | g | — | 0.1 | g | 0.1 | g |

| | | | | |
|---|---|---|---|---|
| Contents per capsule (mg) | 350.0 | 350.0 | 350.0 | 350.0 |

### EXAMPLES 36 TO 39

To a 5 l preparation container equipped with a stirrer, oils and surfactants were added respectively in the amounts shown in Table 11 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in Table 11 and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

The filling content was filled in a medication preparation tank, and charged into a No. 1 hard capsule by 350 mg such that a hard capsule formulation was prepared.

TABLE 11

| | Component | Example 36 | | Example 37 | | Example 38 | | Example 39 | |
|---|---|---|---|---|---|---|---|---|---|
| Main component | Dutasteride | 5 | g | 5 | g | 5 | g | 5 | g |
| | Tadalafil | 50 | g | 200 | g | 50 | g | 50 | g |
| Oil | Glyceryl caprylate/caprate | — | | — | | 275.2 | g | — | |
| | Propylene glycol monolaurate | 3256.9 | g | 2981.9 | g | 2755.4 | g | 3115.9 | g |
| Surfactant | Polyoxyl 40 hardened castor oil | 125 | g | 138 | g | 184.1 | g | — | |
| | Lauroyl Macrogol glyceride | — | | — | | — | | 157 | g |
| | Tocopherol polyethylene | 63 | g | 138 | g | 230.2 | g | 172 | g |
| | glycol succinate | | | | | | | | |
| | Poloxamer 124 | — | | 37 | g | — | | — | |
| Antioxidant | Butylhydroxytoluene | 0.1 | g | 0.1 | g | 0.1 | g | 0.1 | g |

| | | | | |
|---|---|---|---|---|
| Contents per capsule (mg) | 350.0 | 350.0 | 350.0 | 350.0 |

### EXAMPLES 40 TO 44

To a 2 l preparation container equipped with a stirrer, oils and surfactants were added respectively in the amounts shown in Table 12 as below, and the mixture was intensely stirred to prepare a transparent solution in which the surfactants are dissolved. After 5 g of dutasteride was slowly added thereto and completely dissolved, butylhydroxytoluene as an antioxidant was added thereto in the amount shown in Table 12 and the mixture was stirred to prepare a transparent solution. After 50 g of tadalafil was added thereto, the mixture was stirred for 1 hour for dispersion, and further dispersed by a high speed dispersion homogenator for 10 minutes based on the degree of dispersion. Bubbles generated were removed by slowly stirring, such that a composite formulation of dutasteride and tadalafil was prepared.

Separately, a soft capsule shell having the composition shown in Table 4 was prepared. First, concentrated glycerin and a D-sorbitol solution were put into a gelatin preparation tank in the composition shown in Table 4. After purified water, titanium oxide and ferric oxide were put into a separate container and homogeneously suspended with a high-pressure high-speed disperser, the suspension was added in the gelatin preparation tank. Then, gelatin was added in the gelatin preparation tank and wetted while stirring the mixture for 20 minutes. The gelatin preparation tank was vacuumed by a vacuum pump, a heat exchanger was operated to melt the gelatin, and bubbles generated were removed from completely melted gelatin by a vacuum pump, such that a shell filler was prepared.

A medication preparation tank containing the composite formulation content was connected to a stirring tank attached to a soft capsule automatic charger, and 115 mg of the prepared composite formulation content was charged into an oval-type No. 2 soft capsule to a total weight of 235 mg by using a rotary automatic charger. Then, drying and screening processes were performed such that an oral soft capsule formulation was prepared.

TABLE 12

| | | Example | Example | Example | Example | Example |
|---|---|---|---|---|---|---|
| | Component | 40 | 41 | 42 | 43 | 44 |
| Main | Dutasteride | 5 g | 5 g | 5 g | 5 g | 5 g |
| component | Tadalafil | 50 g | 50 g | 50 g | 50 g | 50 g |
| Oil | Propylene glycol monolaurate | 1040.1 g | 1012.8 | 985.4 | 958 | 875.9 g |
| Surfactant | Polyoxyl 40 hardened castor | 21.9 g | 32.8 | 43.8 | 54.8 | 87.6 g |
| | oil | | | | | |
| | Polysorbate 80 | | | | | — |
| | Lauroyl Macrogol glyceride | | | | | — |
| | Tocopherol polyethylene | 14.6 | 21.9 | 29.2 | 36.5 | |
| | glycol succinate | | | | | |
| | Polyoxyl 8 caprylocaproyl | | | | | 58.4 g— |
| | glyceride | | | | | |
| | Poloxamer 124 | 18.3 | 27.4 | 36.5 | 45.6 | 73 g |
| Antioxidant | Butylhydroxytoluene | 0.1 g | 0.1 g | 0.1 g | 0.1 g | 0.1 g |

| | | | | | |
|---|---|---|---|---|---|
| Contents per capsule (mg) | 115.0 | 115.0 | 115.0 | 115.0 | 115.0 |

### COMPARATIVE EXAMPLE 1

A commercially available AVODART® soft capsule of 0.5 mg corresponding to 0.5 mg of dutasteride was used.

### COMPARATIVE EXAMPLE 2

A commercially available Cialis Tab. of 0.5 mg corresponding to 5 mg of tadalafil was used.

### EXPERIMENTAL EXAMPLE 1

### Solubility Test

In order to determine the solubility of dutasteride and tadalafil in oils, the solubilities to soybean oil, castor oil, glycerol monooleate, glycerol caprylate/caprate, propylene glycol monocaprylate and propylene glycol monolaurate were measured. A magnetic bar was placed in a 10 ml, vial, and 3 ml, of oil was added thereto. Then, while the mixture was stirred at room temperature, 100 mg of the main component was added thereto and the mixture was stirred at 500 rpm or higher. After stirring for 24 hours, only a supernatant was taken by a centrifugal separator, and an amount of the main component dissolved in the oil phase was quantitated by using a liquid chromatograph.

TABLE 13

| | Solubility of | Solubility |
|---|---|---|
| | dutasteride | of tadalafil |
| Solvent | (mg/ml) | (mg/ml) |
| Castor oil | 1.50 | 1.03 |
| Soybean oil | 0.00 | 0.24 |
| Glycerol tricaprylate | 0.84 | 0.4 |
| Glycerol caprylate/caprate | 14.34 | 4.13 |
| Glycerol monooleate | 4.58 | 1.11 |
| Propylene glycol dicaprate | 0.77 | 0.27 |
| Propylene glycol dicaprylate | 1.13 | 0.44 |
| Propylene glycol monocaprylate | 20.98 | 4.09 |
| Propylene glycol monolaurate | 9.34 | 1.03 |

<Solubility of Dutasteride According to the Solvent>

Based on the solubility test result according to each oil shown in Table 8, the solubilities of dutasteride and tadalafil in a general oil, i.e., castor oil or soybean oil, are both low. However, in the case of glycerol caprylate/caprate, glycerol monooleate, propylene glycol monocaprylate and propylene glycol monolaurate, which are fatty acid ester derivatives of glycerol and propylene glycol having 8 to 18 carbon atoms, the solubility of dutasteride is 2.5 mg/ml, or higher and the solubility of tadalafil is ⅓ times or less of that of dutasteride. Accordingly, dutasteride may be completely dissolved in the content liquid and tadalafil may be dispersed therein, rather than dissolved, and thus the size of the final formulation may be minimized and patients' compliance with medication may be enhanced.

<Experimental example 2>Severe stability test of soft capsules In order to evaluate the stability in the soft capsule formulation, the formulation according to Example 15 was packaged in an opaque PVDC blister, and then respectively stored at various severe conditions (light: 600 W/m 2 and temperature: 50° C.) to identify the presence of related substances.

TABLE 14

| Condition | Light: 600 W/m² | Temperature: 50° C. |
|---|---|---|
| Example 15 | 6 hours: None | 1 week: None |
| | 2 days: None | 2 weeks: None |
| | 6 days: None | 3 weeks: None |
| | | 4 weeks: None |

<Results of Severe Stability Test of Soft Capsule Formulation>

Based on the test result, as shown in Table 9, it is identified that the composite formulation prepared by one example of the present invention does not generate a related substance when stored under severe conditions in terms of light and temperature and is thus excellent in stability.

### EXPERIMENTAL EXAMPLE 3

### Dissolution Test

The dissolution properties of the composite formulations according to examples 5, 10 and 34 and 14, 15 and 16 were evaluated according to the dissolution test method II in the Korean Pharmacopoeia (10th Edition). A rotation speed was 50 rpm. A 0.3% aqueous solution of sodium lauryl sulfate was used as an eluent for the dissolution test of dutasteride, and a 0.05% aqueous solution of sodium lauryl sulfate was used as an eluent for the dissolution test of tadalafil.

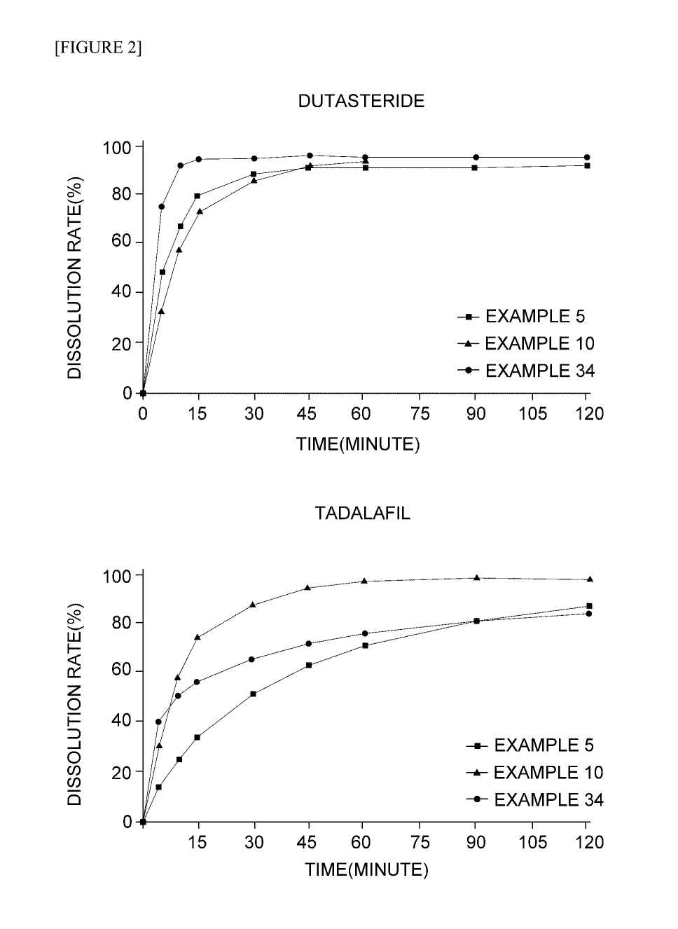

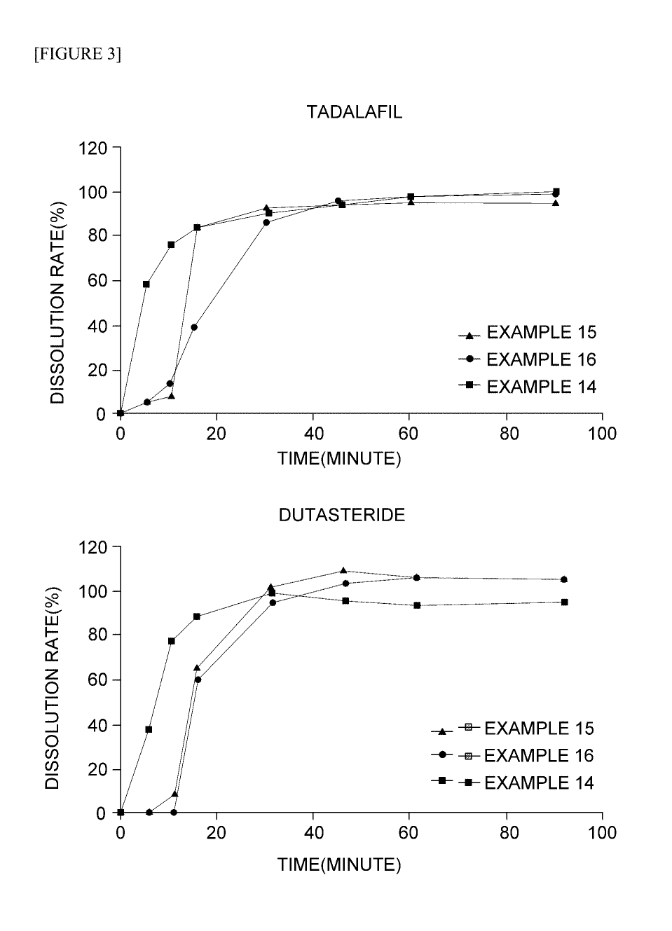

As shown in FIG. 2, the composite formulations of Examples 5, 10 and 34 had the final dissolution rates of 91%, 95% and 94%, respectively, with respect to dutasteride and the final dissolution rates of 85%, 97% and 83%, respectively, with respect to tadalafil.

Further, as shown in FIG. 3, it was identified that the composite formulations of Examples 14, 15 and 16 exhibit an excellent dissolution rate close to 100% within 30 minutes.

### EXPERIMENTAL EXAMPLE 4

### Pharmacokinetic Test

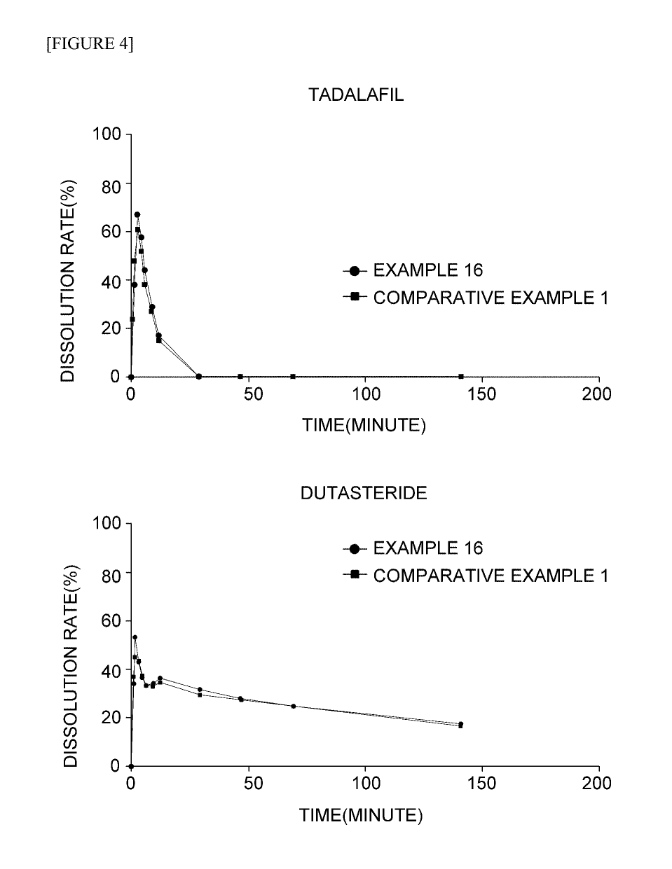

Using a 10-month-old male beagle dog, the pharmacokinetic property of the combined administration of Comparative Example 1 and Comparative Example 2 was compared with that of the medication according to Example 16. The medications were dosed through oral administration, and 3 ml of blood was sampled from the jugular vein through a heparin-treated tube of 10 μl (5 unit) according to time (before administration, 0.5, 1, 2, 3, 4, 6, 8, 24, 48, 72 and 144 hours). The sampled blood was centrifuged at 4° C. and 4,000 rpm for 10 minutes to obtain plasma, which is then analyzed by HPLC. The concentrations of dutasteride and tadalafil in the blood were analyzed and the biological equivalence was compared by calculating the pharmacokinetic parameters. The results are shown in FIG. 4 below.

As shown in FIG. 4, the composite formulation minimized in size according to examples of the present invention was evaluated to be biologically equivalent.

1-18. (canceled)

19. A composite formulation comprising:
dutasteride represented by Formula I;
tadalafil represented by Formula II; and
a fatty acid ester derivative of glycerol or a fatty acid ester derivative of propylene glycol,
wherein the fatty acid ester derivative dissolves the dutasteride and disperses the tadalafil.

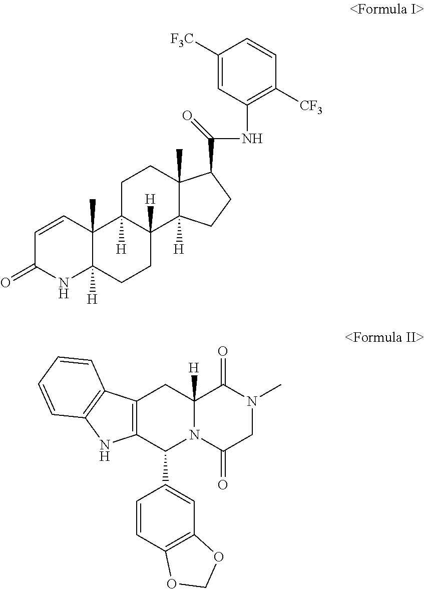

20. The composite formulation as claimed in claim 19, wherein in the fatty acid ester derivative, a fatty acid bonded to glycerol or propylene glycol has 8 to 18 carbon atoms.

21. The composite formulation as claimed in claim 19, wherein the fatty acid ester derivative has a solubility of dutasteride of 1.0 mg/mL or higher, and a solubility of tadalafil is ½ times or less of the solubility of dutasteride.

22. The composite formulation as claimed in claim 19, wherein the fatty acid ester derivative is at least one selected from the group consisting of glycerol caprylate/caprate, glycerol monooleate, propylene glycol monocaprylate and propylene glycol monolaurate.

23. The composite formulation as claimed in claim 19, wherein a content of the fatty acid ester derivative is in a range from 79.0 percent by wt % to 98.95 wt %.

24. The composite formulation as claimed in claim 19, wherein a content of the dutasteride is in a range from 0.05 wt % to 1.5 wt %, a content of the tadalafil is in a range from 1 wt % to 20 wt %, and a content of the fatty acid ester derivative is in a range from 79.0 wt % to 98.95 wt %.

25. An oral capsule formulation comprising the composite formulation of claim 19.

26. The oral capsule formulation as claimed in claim 25, wherein the composite formulation spontaneously forms an emulsion in vivo after administration.

27. The oral capsule formulation as claimed in claim 25, which has a capsule filling amount of the composite formulation is in a range from 100 mg to 400 mg.

28. A composite formulation comprising:
dutasteride represented by Formula I;
tadalafil represented by Formula II;
a fatty acid ester derivative of glycerol or a fatty acid ester derivative of propylene glycol; and
a surfactant,
wherein the fatty acid ester derivative dissolves the dutasteride and disperses the tadalafil.

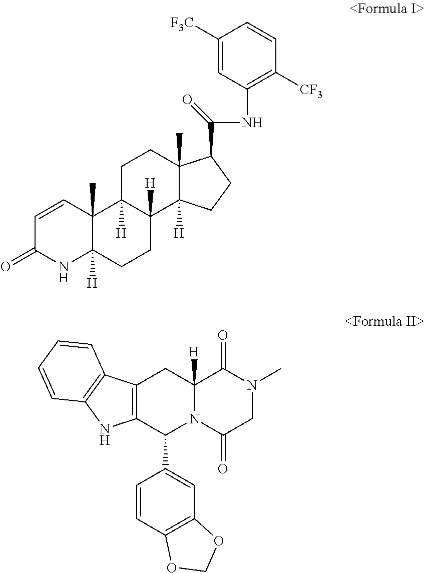

29. The composite formulation as claimed in claim 28, wherein in the fatty acid ester derivative, a fatty acid bonded to glycerol or propylene glycol has 8 to 18 carbon atoms.

30. The composite formulation as claimed in claim 28, wherein the fatty acid ester derivative has a solubility of dutasteride of 1.0 mg/mL or higher, and a solubility of tadalafil is ½ times or less of the solubility of dutasteride.

31. The composite formulation as claimed in claim 28, wherein the fatty acid ester derivative is at least one selected from the group consisting of glycerol caprylate/caprate, glycerol monooleate, propylene glycol monocaprylate, and propylene glycol monolaurate.

32. The composite formulation as claimed in claim 28, wherein the surfactant comprises at least one selected from the group consistin of polyoxyl castor oil, polyoxyl stearic acid, polyoxyl sorbitan fatty acid ester, polyoxyl glyceride, tocopherol polyethylene glycol succinate, and polyoxyethylene-polyoxypropylene copolymers.

33. The composite formulation as claimed in claim 28, wherein a content of the fatty acid ester derivative is in a range from 49.0 wt % to 97.95 wt %.

34. The composite formulation as claimed in claim 28, wherein a content of the surfactant is in a range from 1 wt % to 30 wt %.

35. The composite formulation as claimed in claim 28, wherein a content of the dutasteride is in a range from 0.05 wt % to 1.5 wt %, a content of the tadalafil is in a range from 1 wt % to 20 wt %, a content of the fatty acid ester derivative is in a range from 49.0 wt % to 97.95 wt %, and a content of the surfactant is in a range from 1 wt % to 30 wt %.

36. An oral capsule formulation comprising the composite formulation of claim 28.

37. The oral capsule formulation as claimed in claim 36, wherein the composite formulation spontaneously forms an emulsion in vivo after administration.

38. The oral capsule formulation as claimed in claim 36, which has a capsule filling amount of the composite formulation is in a range from 100 mg to 400 mg.
